(19) United States
(12) Patent Application Publication
(10) Pub. No.: US 2022/0192811 A1
(43) Pub. Date: Jun. 23, 2022

(54) MULTIFUNCTIONAL BIOMESH FOR SURGICAL HERNIA REPAIR

(71) Applicant: Baylor College of Medicine, Houston, TX (US)

(72) Inventors: Ghanashyam ACHARYA, Houston, TX (US); Cryatal S. SHIN, Houston, TX (US); Bradford Glenn SCOTT, Houston, TX (US); Fernando Jose CABRERA, Houston, TX (US)

(21) Appl. No.: 17/596,923

(22) PCT Filed: Jun. 24, 2020
(86) PCT No.: PCT/US20/39393

§ 371 Date: Dec. 21, 2021

Related U.S. Application Data
(60) Provisional application No. 62/867,334, filed on Jun. 27, 2019.

(51) Int. Cl. A61F 2/00; A61L 31/04; A61L 31/16; B33Y 80/00; B33Y 70/00; A61L 31/10

(52) U.S. Cl. CPC A61F 2/0063; A61F 2250/0067; A61L 31/043; A61L 31/16; B33Y 80/00; B33Y 70/00; A61L 31/10; A61L 2300/414; A61L 2300/406; A61L 2300/41; A61L 2300/604; A61L 2300/402; A61L 2400/12; A61F 2210/0004; A61L 31/048

(57) ABSTRACT

Embodiments of the disclosure include methods and compositions related to repair of weakenings or openings in a tissue of an individual, including at least a muscular wall, for example. In specific cases, a mesh comprising phosphate crosslinked poly(vinyl alcohol) polymer (PVA-P) is utilized for such methods, including for hernia repair of any kind. In particular embodiments, one side of the mesh comprises decellularized gel matrix to provide for enhanced tissue healing.

This application claims priority to U.S. Provisional Patent Application Ser. No. 62/867,334, filed Jun. 27, 2019, which is incorporated by reference herein in its entirety.

### TECHNICAL FIELD

Embodiments of the disclosure include at least the fields of surgery, medical devices or equipment, and medicine.

### BACKGROUND

Currently, there are several types of meshes commercially available for surgical hernia repair that include synthetic, biologic, and coated meshes [13-16]. Synthetic non-absorbable meshes made of polypropylene (PP), polyethylene terephthalate (PET or polyester), and expanded poly-tetrafluoroethylene (ePTFE) are in clinical use. A major weakness of nonabsorbable polypropylene based meshes is their high propensity to form adhesions with visceral organs and tissue [17]. Meshes made of ePTFE can reduce visceral adhesion formation, but they lack the ability to promote enough parietal host tissue in-growth. PET meshes exhibit strong tissue in-growth with a less profound foreign body and inflammatory reactions. However, the mechanical stability of the mesh and its susceptibility to infection are the major drawbacks [18]. These nonabsorbable permanent meshes are associated with fistula formation, chronic pain and increased risk for infections [19]. Absorbable meshes are made of polylactic acid, polyglycolic acid, PLGA, or poly(4-hydroxybutyrate). Although these meshes are biodegradable, they lack long-term mechanical strength and stimulate inflammation. Rapid mesh degradation resulting in mechanically unstable collagen formation leading to mesh failure [20-22].

To minimize visceral adhesions to the mesh after its implantation, meshes coated with adhesion barrier agents, such as hyaluronic acid, poliglecaprone, polydioxanone, oxidized regenerated cellulose, and omega-3 fatty acids have been developed [23-25]. The adhesion barrier layers on the surface of coated meshes were able to prevent adhesion formation at 7 days, but this effect diminished in less than 30 days and adhesion formation to the mesh are substantial [26]. Biologic meshes although enhance tissue integration, they must be kept refrigerated and needs to be rehydrated at the time of implantation. Also, these meshes stretch out over time during implantation due to elastin content and are susceptible to degradation by collagenase activity [27-29].

Despite their advantages, none of the presently available meshes have been able to minimize postsurgical complications because of the defective mesh design and incompatible materials used in the mesh, which inflict a substantial cost to the patient in the form of pain, disability, time off from work and procedural costs. The present disclosure satisfies a long-felt need in the art of defective meshes for post-surgical repair.

### BRIEF SUMMARY

The present disclosure is directed to systems, methods and compositions related to weakenings or openings in tissues or organs of a mammal. In particular cases, a surgical mesh (that may also be referred to as a membrane) is provided that has multiple functions. In specific embodiments, the surgical mesh is a biologic mesh or bioscaffold (and may be referred to as “Biomesh” or “Bioscaffold”) that is multifunctional in having the ability to heal the weakening or opening while also having the ability to prevent or reduce adhesion to tissues and/or organs in the body. The multifunctional composite Biomesh of the disclosure is biocompatible and can be used for surgical repair, including at least hernia repair, as an adhesion barrier, as a pelvic floor repair mesh, for cardiothoracic surgery repair, tendon repair, or in breast implant surgery, as examples.

The Biomesh of the disclosure in certain embodiments is bilateral, having two sides each of which are non-identical to the other. The Biomesh is uniquely configured to comprise one side conducive to healing a weakening or opening in tissues and/or organs and a second side conducive to avoiding adhesion to tissues and/or organs that are peritoneal and/or visceral in nature.

In particular embodiments, the disclosure encompasses methods of producing the Biomesh compositions. In specific aspects the Biomesh is comprised at least in part of phosphate crosslinked poly(vinyl alcohol) polymer (PVA-P). Such methods of producing the Biomesh allow for the Biomesh to be degradable in the body at a substantially programmable rate. In particular embodiments, the Biomesh is manufactured by 3D printing.

Certain embodiments of the disclosure encompass methods of using the Biomesh to heal or improve a weakening or opening in a tissue and/or organ of an individual. The weakening or opening in the tissue and/or organ may be the result of natural causes or following a medical procedure in the body, such as surgery of any kind.

In one embodiment, there is a composition comprising a mesh, said mesh comprising phosphate crosslinked poly(vinyl alcohol) polymer (PVA-P). The mesh may comprise a network of structures that are coated with PVA-P and/or the mesh may comprise a network of structures made of PVA-P, in particular cases. The mesh comprises a first side and a second side, and in some cases the first side comprises decellularized tissue matrix. The decellularized tissue matrix may be 3D printed as the first side. The first side of the mesh may be coated with decellularized tissue matrix. In some cases, the surface of the first side is micropatterned, such as the micropattern comprising or substantially comprising a square, rectangular, triangular, pentagonal, or hexagonal pattern. In some cases, the second side is substantially smooth and flat. The mesh may have a tensile strength of greater than 23 kilopascals (kPa). The mesh may be degradable at a rate that is a function of crosslinking of the PVA-P. In specific embodiments, the thickness of the mesh is in a range of about 100 μm-300 μm, 100 μm-200 μm, 100 μm-150 μm, 100 μm-125 μm, 125 μm-300 μm, 125 μm-200 μm, 125 μm-150 μm, 150 μm-300 μm, 150 μm-200 μm, or 200 μm-300 μm.

In some embodiments, there is a method of producing any composition encompassed by the disclosure, comprising the step of manufacturing a mesh comprised of PVA-P or coated with PVA-P. The mesh may be 3D printed as a network of structures comprising PVA-P. In some cases, a printer deposits a pre-polymer solution that polymerizes to PVA-P during the printing process. PVA may be mixed with sodium trimetaphosphate solution prior to printing followed by polymer cross-linking during a drying process. In some cases, the first side of the mesh is 3D printed onto the mesh as decellularized tissue matrix. The mesh may comprise a network of structures coated with PVA-P.

In particular embodiments, there is a method of repairing a weakness or opening in a tissue of an individual, comprising the step of positioning any composition encompassed by the disclosure at the weakness or opening. The tissue may be a muscular wall. In specific embodiments, the method is used for hernia repair, pelvic floor repair, cardiothoracic surgery repair, tendon repair, or breast implant surgery. The composition may comprise a first side and a second side and the first side may comprise decellularized tissue matrix. In specific embodiments, the mesh is positioned such that the first side faces the weakness or opening, and the second side faces a direction opposing the wall. In particular embodiments, any opening is a hernia, and the hernia may be diaphragmatic, inguinal, femoral, umbilical, incisional, epigastric, or hiatal. In at least some cases, there is no adhesion of the composition to one or more peritoneal and/or visceral organs or tissues and/or there is reduced adhesion to one or more peritoneal and/or visceral organs or tissues compared to when using a composition that lacks PVA-P. In particular embodiments, local inflammation is reduced following placement of the composition. The decellularized tissue matrix may stimulate healing of the weakness or opening. In certain embodiments, the mesh degrades in the individual. The mesh may degrade in the individual at a controllable rate of degradation, and the controllable rate of degradation may be controlled by the number of phosphate crosslinking groups in the mesh. Control of sodium trimetaphosphate (STMP) to PVA ratio controls the degree of crosslinking, in specific embodiments.

The foregoing has outlined rather broadly the features and technical advantages of the present disclosure in order that the detailed description that follows may be better understood. Additional features and advantages will be described hereinafter which form the subject of the claims herein. It should be appreciated by those skilled in the art that the conception and specific embodiments disclosed may be readily utilized as a basis for modifying or designing other structures for carrying out the same purposes of the present designs. It should also be realized by those skilled in the art that such equivalent constructions do not depart from the spirit and scope as set forth in the appended claims. The novel features which are believed to be characteristic of the designs disclosed herein, both as to the organization and method of operation, together with further objects and advantages will be better understood from the following description when considered in connection with the accompanying figures. It is to be expressly understood, however, that each of the figures is provided for the purpose of illustration and description only and is not intended as a definition of the limits of the present disclosure.

### BRIEF DESCRIPTION OF THE DRAWINGS

For a more complete understanding of the present disclosure, reference is now made to the following descriptions taken in conjunction with the accompanying drawings.

FIGS. 1A-1B provide schematics depicting surgical hernia repair by the implantation of a mesh. (1A) A hernia site depicting the hole in the abdominal wall and the protrusion of small intestine. (1B) Surgical hernia repair by implanting a mesh. FIGS. 1C-1E provide schematics depicting the mechanism of modulation of peritoneal adhesions formation by a Biomesh. (1C) Damage to the mesothelial layer of the peritoneum wherein positively charged pro-inflammatory cytokines have been secreted. (1D) Adhesions formation by the mesothelium connecting the peritoneal layer and the visceral organs. (1E) Implantation of a negatively charged Biomesh to adsorb the positively charged proinflammatory cytokines from the damaged peritoneum after surgical trauma.

FIG. 2A shows a schematic of one embodiment of a 3D-printed Biomesh (top view). FIGS. 2B-2D show a Biomesh design of one embodiment generated by BioCad™ for printing. In some embodiments, each run is composed of sequential printing of the arrays of FIGS. 2B-2D.

FIG. 3 illustrates PVA crosslinking reaction with sodium trimetaphosphate (STMP) to form phosphate crosslinked PVA-P.

FIGS. 4A-4C represent aspects of 3D-fabricated multifunctional Biomesh. (4A) 3D-fabricated Biomesh. (4B) Biomesh printed with decellularized gel matrix on the parietal side. (4C) A plot depicting the tensile strengths of different Biomesh.

FIGS. 5A-5G demonstrate some embodiments of Biomesh fabrication and characterization. (5A) 3D-printed Biomesh. (5B-5D) 3D-printed Biomesh pliability. (5E) A plot depicting the elastic moduli of different Biomesh. (5F) Consistent suture retention capacity of Biomesh after several stress-strain cycles. (5G) A plot depicting the zeta potential of different Biomesh.

FIGS. 6A-6G demonstrate aspects of Biomesh acting as an inflammatory cytokine capturing surface. (6A) Schematic presenting the in vitro strategy for the inflammatory cytokine capture by the Biomesh. (6B-6G) Overlays of bright field and fluorescence, and fluorescence confocal micrographs demonstrating: (6B) nonbinding of the antibody beads in the absence of cytokines on the Biomesh surface; (6C) TNF-a antibody beads binding to the TNF-a adsorbed on the Biomesh surface; (6D) IL-1a antibody beads bound to the IL-1a bound to the Biomesh surface; (6E) IL-6 antibody beads bound to the IL-6 adsorbed on the Biomesh surface; (6F) MIP-1a antibody beads bound to the MIP-1a adsorbed on the Biomesh surface; and (6G) VEGF antibody beads bound to the VEGF adsorbed on the Biomesh surface.

FIG. 7 demonstrates aspects of Biomesh effectively modulating the levels of proinflammatory cytokines, proinflammatory matrix metalloproteases, profibrotic factors, and proangiogenic factors in the hernia repair site in a first rat ventral hernia model. FIG. 7 shows RT-PCR studies presenting the expression levels of IL-1β, IL-8, MMP-8, MMP-12, TGF-β1, TGF-β2, VEGF-A, and COL-1a. N=3 (5 animals per group).*P<0.01;**P<0.01; ***P<0.001. All error bars represent standard deviation from the mean. Left bars=PROLENE® mesh; middle bars=Biomesh; and right bars=PVA-P coated PROLENE®mesh.

FIG. 8 further demonstrates aspects of Biomesh effectively modulating the levels of proinflammatory cytokines, proinflammatory matrix metalloproteases, profibrotic factors, and proangiogenic factors in the hernia repair site in a second rat ventral hernia model. FIG. 8 shows qPCR studies presenting the expression levels of IL-1β, IL-6, IL-8, TNF-α, MMP-8, TGF-β1, and VEGF. All error bars represent standard deviation from the mean.*P<0.05, **P<0.01, ***P<0.001,****P<0.0001. Left bars=PROLENE® mesh; middle bars=Biomesh; and right bars=PVA-P coated PROLENE®mesh.

FIGS. 9A-9I demonstrate aspects of Biomesh effectively preventing adhesions after its surgical implantation in ventral hernia rat model. (9A) PROLENE® mesh; (9B) Biomesh; (9C) PROLENE® mesh coated with PVA-P. After two weeks implantation: (9D) PROLENE® mesh; (9E) Biomesh; (9F) PROLENE® mesh coated with PVA-P. After 4 weeks implantation: (9G) PROLENE® mesh; (9H) Biomesh; (9I) PROLENE® mesh coated with PVA-P.

FIGS. 10A-10C demonstrate aspects of Biomesh effectively preventing peritoneal adhesions. (10A) PROLENE® mesh demonstrating peritoneum adhering to its surface (black arrows indicating adhesions); (10B) Biomesh; and (10C) PVA-P coated PROLENE® mesh developed no adhesions; Red arrows in the images indicate the mesh location.

FIGS. 11A-11F demonstrate aspects of the surface charge effect of Biomesh on adhesion formations: (11A) PROLENE mesh, (11B) Biomesh, (11C) Neutral Biomesh, (11D) Polylysine coated positively charged Biomesh, (11E) Fibronectin coated Biomesh, (11F) Gelatin coated Biomesh. FIG. 11G shows that negatively charged Biomesh effectively modulates the levels of proinflammatory cytokines, profibrotic factors, and proangiogenic factors. FIG. 11G shows qPCR analysis of surface charge effects of Biomesh on expression levels of IL-1β, IL-6, TNF-α, TGF-β1, VEGF, ICAM1, COL1a1, and COL3a1. All error bars represent standard deviation from the mean.*P<0.05, **P<0.01,***P<0.001,****P<0.0001. Far left bars=PROLENE® mesh; left middle bars=Neutral Biomesh; right middle bars=Biomesh, and Far right bars=PVA-P coated PROLENE® mesh.

FIGS. 12A-12B demonstrate aspects of Biomesh stimulating the hernia healing process. Parietal side of the hernia repair one month after the implantation of (12A) polypropylene mesh; and (12B) Biomesh. Blue knots around the wound are sutures.

FIGS. 13A-13B demonstrates aspects of macroscopic grading of the extent of postoperative adhesions after the implantation of Biomesh in rat ventral hernia model. (13A) Adhesions Extent at 2 weeks. (13B) Adhesions Extent at 4 weeks. Adhesions Extent was graded on a scale of 0 to 4 (0=absence of adhesions; 1=0-25% of the mesh is covered with adhesions; 2=25-50% of the mesh is covered with adhesions; 3=50-75% of the mesh is covered with adhesions; 4=75-100% of the mesh is covered with adhesions).

FIGS. 14A-14B demonstrates aspects of macroscopic grading of the tenacity of postoperative adhesions after the implantation of Biomesh in rat ventral hernia model. (14A) Adhesions Tenacity at 2 weeks. (14B) Adhesions Tenacity at 4 weeks. Adhesion tenacity was graded on a scale of 0 to 3 (0=absence of adhesions; 1=adhesions easily fall apart; 2=adhesions need to be pulled to separate; 3=adhesions require sharp dissection to separate from the mesh).

FIGS. 15A-15B demonstrates aspects of macroscopic grading of the type of postoperative adhesions after the implantation of Biomesh in rat ventral hernia model. (15A) Adhesions Type at 2 weeks. (15B) Adhesions Type at 4 weeks. Adhesion type was graded on a scale of 0 to 3 (0=no adhesions; 1=filmy; 2=dense; 3=capillaries present).

### DETAILED DESCRIPTION

As used herein, the terms “or” and “and/or” are utilized to describe multiple components in combination or exclusive of one another. For example, “x, y, and/or z” can refer to “x” alone, “y” alone, “z” alone, “x, y, and z,” “(x and y) or z,” “x or (y and z),” or “x or y or z.” It is specifically contemplated that x, y, or z may be specifically excluded from an embodiment.

As used herein, the term “about” or “approximately” refers to a quantity, level, value, number, frequency, percentage, dimension, size, amount, weight or length that varies by as much as 30, 25, 20, 25, 10, 9, 8, 7, 6, 5, 4, 3, 2 or 1% to a reference quantity, level, value, number, frequency, percentage, dimension, size, amount, weight or length. In particular embodiments, the terms “about” or “approximately” when preceding a numerical value indicates the value plus or minus a range of 15%, 10%, 5%, or 1%. With respect to biological systems or processes, the term can mean within an order of magnitude, preferably within 5-fold, and more preferably within 2-fold, of a value. Unless otherwise stated, the term ‘about’ means within an acceptable error range for the particular value.

It is specifically contemplated that any limitation discussed with respect to one embodiment of the disclosure may apply to any other embodiment of the invention. Furthermore, any composition of the disclosure may be used in any method of the invention, and any method of the invention may be used to produce or to utilize any composition of the invention. Aspects of an embodiment set forth in the Examples are also embodiments that may be implemented in the context of embodiments discussed elsewhere in a different Example or elsewhere in the application, such as in the Summary of Invention, Detailed Description of the Embodiments, Claims, and description of Figure Legends.

In keeping with long-standing patent law convention, the words “a” and “an” when used in the present specification in concert with the word comprising, including the claims, denote “one or more.” Some embodiments of the disclosure may consist of or consist essentially of one or more elements, method steps, and/or methods of the disclosure. It is contemplated that any method or composition described herein can be implemented with respect to any other method or composition described herein and that different embodiments may be combined.

Throughout this specification, unless the context requires otherwise, the words “comprise”, “comprises” and “comprising” will be understood to imply the inclusion of a stated step or element or group of steps or elements but not the exclusion of any other step or element or group of steps or elements. By “consisting of” is meant including, and limited to, whatever follows the phrase “consisting of.” Thus, the phrase “consisting of” indicates that the listed elements are required or mandatory, and that no other elements may be present. By “consisting essentially of” is meant including any elements listed after the phrase, and limited to other elements that do not interfere with or contribute to the activity or action specified in the disclosure for the listed elements. Thus, the phrase “consisting essentially of” indicates that the listed elements are required or mandatory, but that no other elements are optional and may or may not be present depending upon whether or not they affect the activity or action of the listed elements.

Reference throughout this specification to “one embodiment,” “an embodiment,” “a particular embodiment,” “a related embodiment,” “a certain embodiment,” “an additional embodiment,” or “a further embodiment” or combinations thereof means that a particular feature, structure or characteristic described in connection with the embodiment is included in at least one embodiment of the present invention. Thus, the appearances of the foregoing phrases in various places throughout this specification are not necessarily all referring to the same embodiment. Furthermore, the particular features, structures, or characteristics may be combined in any suitable manner in one or more embodiments.

The term “subject,” as used herein, generally refers to an individual in need of therapeutic intervention, including an individual that has a hernia, as one example. A subject can be an animal. The subject can be any animal subject that is an object of a method or material, including mammals, e.g., humans, laboratory animals (e.g., primates, rats, mice, rabbits), livestock (e.g., cows, sheep, goats, pigs, turkeys, and chickens), household pets (e.g., dogs, cats, and rodents), horses, and transgenic non-human animals. The subject can be a patient, e.g., have or be suspected of having a medical condition in need, such as a subject undergoing surgery of some type. The term “individual” may be used interchangeably, in at least some cases. The “subject” or “individual”, as used herein, may or may not be housed in a medical facility and may be treated as an outpatient of a medical facility. An individual may comprise any age of a human or non-human animal and therefore includes both adult and juveniles (i.e., children) and infants and includes in utero individuals. It is not intended that the term connote a need for medical treatment, therefore, an individual may voluntarily or involuntarily be part of experimentation whether clinical or in support of basic science studies.

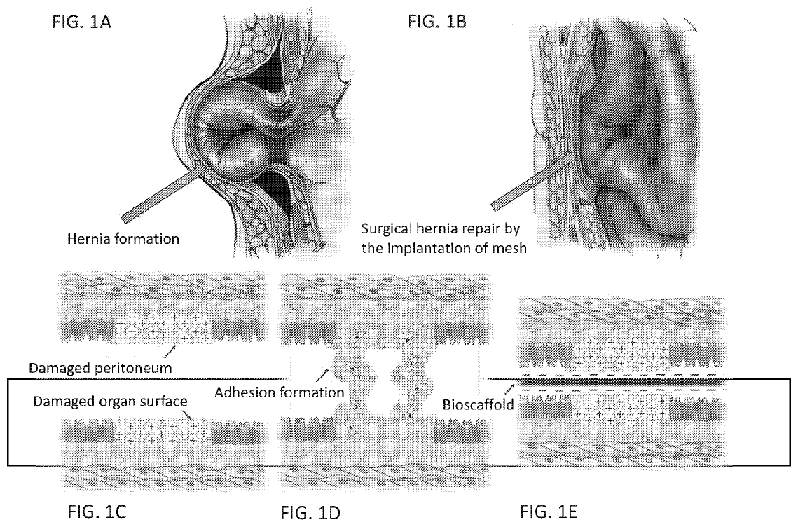

Development of a multifunctional Biomesh is of critical importance in improving the patient outcomes after surgical repair, including hernia repair. The abdominal wall encloses and protects the organs in the abdominal cavity. However, weak areas in the abdominal wall because of injury or surgical procedures cause abnormal protrusion of organs through these defects, thus resulting in a hernia [1]. Each year, over 400,000 incisional hernia repair surgeries are performed with a cost of ˜$15 billion to the US healthcare expenditure. [30]. More than 80% of these hernia repairs involve the use of mesh products [2,3]. Hernia repair is performed by the surgical implantation of a polymer mesh to firmly support and reinforce the weakened tissue around the hernia that is no longer able to retain its shape or physical function (FIG. 1). The implanted mesh reduces pressure on the damaged abdominal wall near the hernia site and facilitates healing process [2,3].

Presently more than 70 different types of meshes based on synthetic, biologic, and coated materials are in use for surgical hernia repair [2,3]. Despite their advantages, none of these meshes have been able to minimize the adverse complications [4-7]. Surgical hernia repair often results in visceral adhesions, which are fibrous tissues that develop from the underlying serosal membrane and the underlying organs and attach to the implanted mesh. These adhesions stick and grow on the hernia mesh causing chronic pain, bowel obstruction, fistula infertility, poor quality of life, and require revision surgery [4-7]. Chronic pain lasting for more than 3 to 6 months after surgery will be severe enough to bring a change in lifestyle, to cause a severe handicap in ordinary activities, or make life unbearable in up to 60% of the cases [7].

Dysejaculation: in the case of inguinal hernias, excruciating and searing pain during ejaculation due to the movement or migration of mesh penetrating the layers of the spermatic cord and the vas deferens. In extreme cases, mesh can invade the entire spermatic cord causing pain. Infertility can also be a problem in bilateral inguinal hernias. These effects of mesh are irreversible [4-7]. Hernia recurrence: to be effective the abdominal wall must remain flexible as the torso twists and rotates. As the mesh hardens, it becomes more rigid and has little tensile strength. This can cause the hernia repair to fail. In addition, absorbable and biological meshes, designed to minimize scarring and foreign body reactions, when used independently, have little long-term value [24,25]. Recurrence rates have approached 100% for these products. Complicated surgery to remove the mesh: after implantation because of adhesion formation, the mesh becomes embedded in the surrounding tissues and organs, and its removal can involve risky surgery with uncertain results and consequences. Although there are inherent risks in any surgery, there are no comparable risks in hernia mesh removal, where the mesh must be peeled off a bladder, colon, a spermatic cord or a major blood vessel.

Considering the devastating complications caused by the presently available meshes, the present disclosure provides a multifunctional Biomesh for repair or tissue weakenings or openings and also that can (1) prevent or at least reduce the number or amount or extent of peritoneal adhesion formation; (2) stimulate rapid tissue integration; and/or (3) prevent recurrence and revision surgery.

### I. Compositions

A. Considerations in Biomesh Design

Although hernia meshes have been in use for several years, mesh related complications such as visceral adhesions, bowel obstruction, chronic pain and infection are a major concern and adversely affect the clinical outcomes. Causative factors for adverse complications arising due to surgical mesh implantation are chronic inflammatory responses and poor mesh-tissue integration. The adverse effects triggered by the implanted hernia mesh are related to the chemical and structural nature of the mesh itself.

1. Inflammation. Implantation of a hernia mesh stimulates an inflammatory reaction that causes the formation of visceral adhesions. There is a strong association between visceral adhesions and small bowel obstruction, infertility, and chronic pain, often requiring a revision surgery [4-6]. Therefore, the multifunctional surgical mesh (Biomesh) of the disclosure is fabricated with novel noninflammatory materials that after surgical implantation are inert, non-immunogenic and prevent adhesion to unintended target tissue(s), including visceral adhesion formation.

2. Mesh Materials. To surmount the mesh-related complications, meshes coated with adhesion barrier materials, absorbable and biological meshes have been developed. All these efforts have met with limited success. The adhesion barrier coatings on the meshes dissolve, leaving the same polypropylene to cause its complications, and many of these coatings designed to protect the mesh have in themselves caused life threatening infections. In coated meshes, as the mesh coating deteriorates during its absorption, hernia recurrence was observed in virtually all cases. The Biomesh of the disclosure, because of its intrinsic non-inflammatory nature and longer degradation time minimizes mesh-related complications, such as after a surgical procedure, including at least hernia repair.

3. Mesh Design. Most commonly used meshes are highly porous as they are made using synthetic polymer fibers. During the healing process, fibroblasts and collagen grow into the mesh through the pores, forming a large layer of scar tissue. Mesh can also adhere to surrounding tissue, nerves and organs. As the scar tissue shrinks, so does the mesh, creating a hard, fibrous mass with nerves embedded within it, thus causing chronic severe pain. Over time, the mesh also hardens and become less flexible to the point where explanted mesh samples have become hardened plastic. The Biomesh of the disclosure may be designed to have a flat smooth surface on the visceral side to prevent adhesion formation and a micropatterned surface on the parietal side to stimulate cell adhesion and proliferation. Because of the nonporous and non-inflammatory nature of the Biomesh of the disclosure, the tissue adhesions are prevented or at least reduced on the visceral side, and this minimizes chronic pain.

B. General Embodiments of the Compositions

In general embodiments of the disclosure, the multifunctional Biomesh design utilizes: (1) the use of a PVA-P composite, (2) the use of decellularized gel matrix, and (3) optionally Biomesh fabrication by 3D-bioprinting strategy.

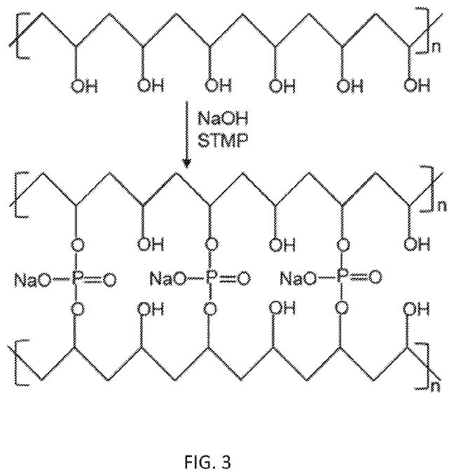

With respect to a phosphate crosslinked PVA-P composite, the PVA-P composite in at least some cases is synthesized by in situ crosslinking of PVA with sodium trimetaphosphate (STMP) (FIG. 3). The crosslinking density of PVA-P composite can be optimized to provide the required tensile strength and mechanical stability to the Biomesh. Rapid deterioration of the tensile strength of a mesh could potentially lead to hernia recurrence or a poor functional result. Hence, the PVA-P composite in the Biomesh is optimized to possess an optimum tensile strength necessary to withstand the stresses placed on the abdominal wall. In a healthy adult, coughing or jumping will generate a peak intra-abdominal pressure of 23 kPa. Hence, the Biomesh in at least some embodiments possesses a tensile strength of >23 kPa to withstand the abdominal stress once implanted. Based on this data, the Biomesh is fabricated to possess a physiological tensile strength of at least 23 kPa and above to provide effective and functional support to the hernia site, in at least some embodiments.

The PVA-P composite possesses an intrinsic net negative charge on its surface and behaves as an efficient adhesion barrier. The PVA-P composite is non-inflammatory and prevents visceral adhesions, seroma, and hematoma formation around the Biomesh upon surgical implantation. The Biomesh in at least some embodiments slowly degrades after a period of time following implantation (for example, about 1 yr, 1.5yrs, 2 yrs, 2.5 years, and so forth), by then the hernia regains strength and heals completely.

With respect to decellularized gel matrix, in at least some embodiments the visceral side of the Biomesh is 3D-printed with decellularized gel matrix on an opposing side of the Biomesh from the visceral side to provide a special microenvironment suitable for rapid healing of the weakening or opening (e.g., hernia, abdominal wall injuries or surgical incisions). One rationale for using decellularized gel matrix in the Biomesh is to accelerate vascularization of the Biomesh, improve soft tissue regeneration, reduce postoperative morbidity, and enhance the abdominal wall healing process [8]. Decellularized tissue gel is a natural source of growth factors such as vascular endothelial growth factor (VEGF), basic fibroblast growth factor (bFGF), transforming growth factor beta (TGF-β), platelet derived growth factor (PDGF), collagen, and/or angiopoietin. Decellularized gel matrix is generated from an organ by a decellularization process. It is temperature-sensitive hydrogel, in at least some cases. In some embodiments, it will be printed on one side of the Biomesh, such as the side of the Biomesh that will be facing the muscle. In some embodiments, it is a coating on the Biomesh and does not provide any substantial structural support to it. However, in some embodiments it is printed on the Biomesh to stimulate angiogenesis and the healing process. The growth factors from the decellularized tissue gel are involved in key stages of wound healing and regenerative processes including chemotaxis, proliferation, differentiation, and angiogenesis [31]. For example, bFGF is a potent mitogen that can stimulate the growth and differentiation of a wide variety of cell types and it plays an important role in tissue remodeling, wound healing and neovascularization [28]. In addition to growth factors, the decellularized gel matrix also comprises integrins, fibronectin, vitronectin, etc., that are useful in stimulating angiogenesis, increase fibroblast cell differentiation, and promote soft tissue regeneration and wound healing. The Biomesh in at least some cases may be evaluated for its tissue regeneration efficacy in chronic hernia rat model.

In at least some embodiments, part or all of the Biomesh is absorbable and/or biodegradable yet still has mechanical strength and does not stimulate inflammation at all or at least to the level of surgical meshes known in the art. The Biomesh of the disclosure may in specific cases have one or more of the following attributes: less susceptible to degradation, for example by collagenase; lacks requirement for refrigeration and/or rehydration prior to use; has a reduced tendency to form adhesions, including with visceral organs and/or tissue; able to promote parietal host tissue in-growth; is mechanically stable and less risk for infection than presently used meshes; the surface has a net negative charge; is nonporous; and/or is hydrophilic. The Biomesh may be white or it may be colored.

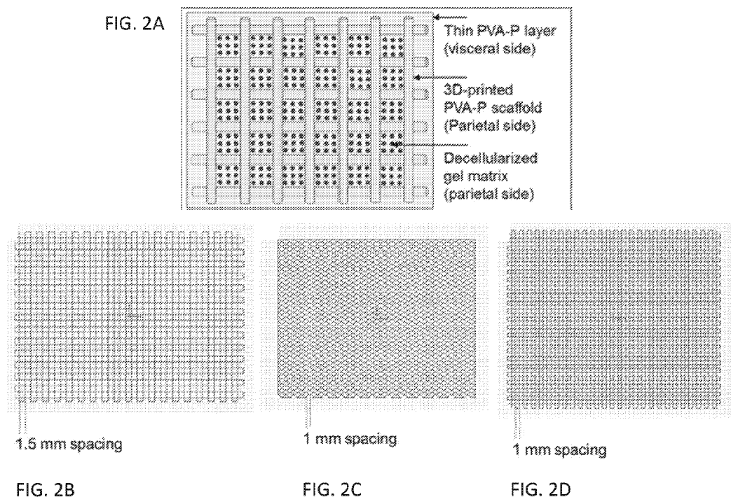

In particular embodiments, the composition of the Biomesh has at least part of it being micropatterned, and in specific embodiments the micropatterning is on the parietal side that comprises the decellularized gel matrix. As used herein, the term “micropatterned” refers to the 3D-printed pattern of lines of the mesh that in at least specific cases generally are in a square or rectangular pattern (see FIG. 2, for example). In particular cases the Biomesh comprises a network of structures (lines, threads, etc.), and in at least some cases each line is approximately at least or no more than 100 μm to 1 mm thick. The range of thickness may be from 100-1000, 100-900, 100-800, 100-700, 100-600, 100-500, 100-400, 100-300, 100-200, 100-150, 200-1000, 200-900, 200-800, 200-700, 200-600, 200-500, 200-400, 200-300, 300-1000, 300-900, 300-800, 300-700, 300-600, 300-500, 300-400, 400-1000, 400-900, 400-800, 400-700, 400-600, 400-500, 500-1000, 500-900, 500-800, 500-700, 500-600, 600-1000, 600-900, 600-800, 600-700, 700-1000, 700-900, 700-800, 800-1000, 800-900, or 900-1000 μm. The thickness may be at least or no more than 100, 150, 200, 300, 400, 500, 600, 700, 800, 900, or 1000 μm.

Embodiments of the disclosure provide for an adhesion barrier comprising phosphate crosslinked poly(vinyl alcohol) polymer (PVA-P). The barrier may comprise a network of structures and the network may be coated with PVA-P, and/or the barrier may comprise a network of structures made of PVA-P. In particular embodiments, the barrier comprises a first side and a second side. The first side may comprise decellularized tissue matrix. The decellularized tissue matrix may or may not be 3D printed as the first side. The second side may lack decellularized tissue matrix.

Embodiments of the disclosure include phosphate crosslinked PVA polymer (PVA-P) as an adhesion barrier. Certain embodiments of the disclosure include a PVA-P membrane that upon implantation suppresses local inflammation and prevents peritoneal and visceral tissue adhesion formation; the disclosure encompasses a PVA-P-coated commercial hernia mesh having the same characteristics. The PVA-P membrane and coated mesh may be used for diaphragmatic hernia repair and pelvic floor repair, in at least some cases. In certain aspects, on one side of the PVA-P membrane, decellularized gel matrix is placed or deposited, such as by 3D-printing. The PVA-P membrane and coated mesh printed with decellularized gel matrix on the parietal side stimulates the wound healing process and the non-coated visceral side that lacks decellularized gel matrix prevents adhesion formation.

In this disclosure, a multifunctional Biomesh with a phosphate crosslinked polyvinyl alcohol polymer (PVA-P) composite is provided by any means and for any purpose. It may be fabricated by any manner, but in specific cases it is fabricated by 3D bioprinting and decellularized tissue gel is printed on the parietal side of the mesh to promote rapid tissue integration and healing [8]. The Biomesh possesses an optimal tensile strength to provide the required mechanical support to the hernia site. This Biomesh is novel at least because the PVA-P polymer is noninflammatory, prevents adhesion formation on the visceral side, while the parietal side containing decellularized tissue matrix stimulates peritoneal regeneration and healing of the hernia [8]. The Biomesh in specific embodiments may be programmed to degrade after a particular period of time, such as about 2 years, while the hernia will regain strength and heal completely. The Biomesh prevents the postoperative adhesion formation and stimulates rapid tissue integration. The Biomesh after surgical implantation enhances the abdominal wall healing process and reduces postoperative morbidity, chronic pain, and hernia recurrence. In particular embodiments, the Biomesh has a thickness on the order of about 100 μm-300 μm. A multifunctional Biomesh provides a solution to an unmet need in surgical hernia repair with a positive clinical outcome and improve the overall quality of patient's life.

A multifunctional Biomesh with a phosphate crosslinked polyvinyl alcohol polymer (PVAP) composite was developed by 3D bioprinting, and decellularized tissue gel will be printed on the parietal side of the mesh to promote rapid tissue integration and healing, in at least some cases. The Biomesh possesses an optimal tensile strength to provide the required mechanical support to the hernia site. The Biomesh comprising PVAP polymer is noninflammatory, prevents visceral adhesion formation on the visceral side, while the parietal side containing decellularized tissue matrix stimulates peritoneal regeneration and healing of the hernia. In specific cases, the Biomesh is programmed to degrade after about 1, 1.5, 2, or more years while the weakening or opening will regain strength and heal completely. The Biomesh prevents the postoperative adhesion formation and stimulates rapid tissue integration. The Biomesh after surgical implantation will enhance the abdominal wall healing process and reduce postoperative morbidity, chronic pain, and hernia recurrence. The disclosure provides a multifunctional Biomesh that satisfies unmet needs in surgical hernia repair resulting in a positive clinical outcome and improve the overall quality of patient's life. A multifunctional composite Biomesh is novel and innovative, and presently such prosthesis is not available.

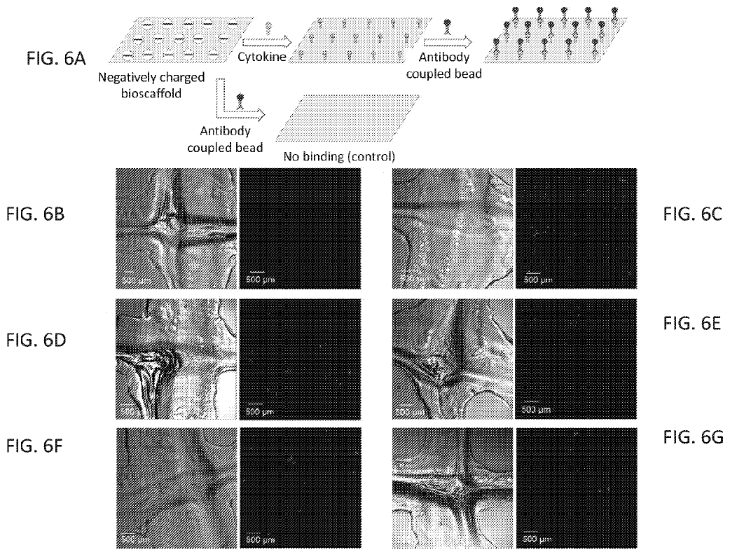

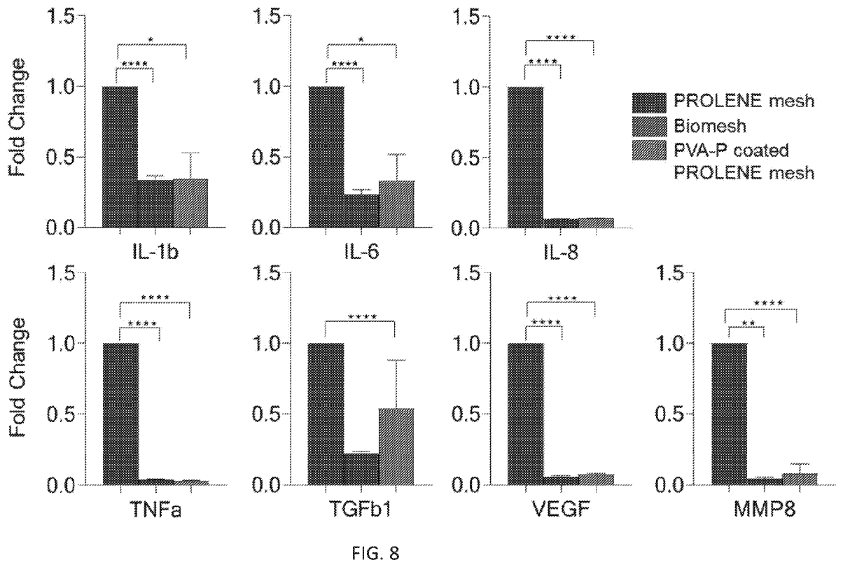

The multifunctional Biomesh is incorporated with several innovative attributes, in at least some cases. (1) Biomesh fabrication by 3D-bioprinting. The multifunctional Biomesh will be fabricated by 3D-biopringing strategy, which enables the integration of in situ crosslinked PVA-P scaffold with decellularized gel matrix [8]. 3D-bioprinting strategy enables the fabrication of PVA-P scaffold of required thickness, tensile strength, flexibility and degradation life time. 3D-bioprinting provides a multifunctional, patient specific Biomesh fabrication for surgical hernia repair. (2) Biomesh is noninflammatory and prevents visceral adhesion formation. The PVA-P composite possesses an intrinsic net negative charge on its surface and behaves as an efficient adhesion barrier. The PVA-P composite is noninflammatory and prevents visceral adhesions, seroma, and hematoma formation around the Biomesh upon surgical implantation. (3) Biomesh provides special microenvironment for abdominal wall regeneration. The visceral side of the Biomesh will be 3D-printed with decellularized gel matrix on an opposing side of the Biomesh from the visceral side. The decellularized gel matrix is a natural source of growth factors and integrins that accelerate angiogenesis, fibroblast cell differentiation, and collagen deposition. Hence, the Biomesh provides a special microenvironment suitable for rapid healing of the damaged abdominal wall. (4) Biomesh is biodegradable. The degradation rate (life time) of the 3D-printed Biomesh can be programmed by controlling the phosphate crosslinking density, PVA molecular weight and scaffold thickness. The phosphate crosslinks in PVA-P will be programmed to maintain the tensile strength of the Biomesh for two years followed by slow degradation, by then the damaged abdominal wall (hernia site) is expected to regain strength and heal completely. (5) Biomesh do not shrink or curl during the wound healing process. Because the Biomesh is nonporous and highly hydrophilic, it will not shrink or curl after implantation (FIGS. 6 and 8). In particular embodiments, although the Biomesh is nonporous, the decellularized gel matrix allows vascularization of the Biomesh. The Biomesh retains its original shape during the wound healing process thus minimizing chronic pain, as experienced with presently available hernia repair meshes. (6) Biomesh is clinically translatable. Because of its simple, easy, and inexpensive fabrication process, and the use of PVA which is safe, noninflammatory, nonimmunogenic and is presently in clinical use, the Biomesh upon development will be clinically translatable. (7) Biomesh improves the clinical outcomes. 3D-printed Biomesh can be fabricated in real time and could serve as a patient-specific and cost-effective alternative to commercial, off-the-shelf meshes. Biomesh can be tailored to meet intraoperative measurements, patient anatomy, and specific situations that could stimulate the healing process. 3D-printed Biomesh enables the development of multifunctional, prosthesis for surgical hernia repair thus improving the clinical outcomes.

In particular embodiments, the Biomesh has a programmed life time because it has been manufactured for the purpose of having a specific degradation rate to allow for degradation to occur following a long enough duration after implantation to allow for sufficient healing of the weakening or opening. For example, the Biomesh may be generated with the purpose of having a certain degree of phosphate crosslinking groups, and this certain degree is known or determined to prevent degradation occurring prematurely.

In particular embodiments, the disclosure provides a multifunctional, efficacious, composite Biomesh developed for the purpose of surgical hernia repair. The Biomesh is configured to function as an adhesion barrier on the visceral side to prevent adhesions, while the parietal side 3D-printed with decellularize gel matrix provides a special microenvironment suitable for cell adhesion, proliferation, and angiogenesis, thus, facilitating rapid healing of the hernia. In addition, the Biomesh provides the necessary mechanical support to the site of a weakening or opening, such as with a hernia. The Biomesh minimizes the hernia recurrence and revision surgery and improves clinical outcomes.

### II. Production of a Multifunctional Biomesh

Presently available prefabricated commercial meshes often need to be cut with a scissors to match the hernia size and shape of the patient, exposing the naked sides of the mesh to the tissue at the site of implantation, thus stimulating an adverse inflammatory response followed by adhesion formation and other mesh-related complications. 3D-bioprinting as with embodiments of the disclosure provides patient-specific surgical mesh fabrication for tissue repair, including at least hernia repair.

3D-bioprinting also enables the fabrication of Biomesh with a required thickness and life-time of decellularized gel matrix on parietal side to stimulate abdominal wall tissue regeneration after surgical implantation. Furthermore, based on the condition of the abdominal wall, such as the presence of infections, inflammation, hematoma, etc., in particular embodiments the Biomesh can be incorporated with controlled release drug delivery nanoparticles for the local delivery of antibiotics, analgesics, and/or steroids, for example to eliminate postoperative complications. 3D-printed Biomesh tailored to intraoperative measurements, patient anatomy, specific situations, and patient comorbidities stimulates the healing process and improves clinical outcomes. 3D-printed Biomesh serves as a cost-effective alternative to commercial, off-the-shelf meshes. 3D-fabrication strategy is simple, inexpensive and the Biomesh is more efficacious than the presently available surgical (including hernia) meshes. 3D-printed Biomesh enables the fabrication multifunctional, patient-specific prosthesis for surgical hernia repair, thus improving the clinical outcomes.

The multifunctional Biomesh may be fabricated by 3D-bioprinting strategy, which enables the integration of PVA-P mesh scaffold with decellularized gel matrix. The 3D-bioprinting strategy enables the fabrication of customized meshes with required thickness, tensile strength, flexibility, and it also enables the reinforcement areas to be generated as suture points. The degradation rate of the 3D-printed Biomesh can be programmed by controlling the crosslinking density and mesh thickness. For example, the degradation rate of Biomesh is dependent upon the number of phosphate crosslinking groups in the Biomesh. By controlling the PVA to STMP concentration, the approximate percentage of phosphate crosslinks can be estimated. Also, the mechanical strength of the Biomesh is dependent on the crosslinking density. By quantifying the number of phosphate crosslinking groups, the mechanical strength of the Biomesh and also degradation rates can be quantified.

In one embodiment, there is a method of producing any composition encompassed by the disclosure by manufacturing a mesh comprised of PVA-P and/or coated with PVA-P. In specific cases, the mesh is 3D printed as a network of structures comprising PVA-P. A suitable printer may deposit a pre-polymer solution that polymerizes to PVA-P during the printing process, in certain cases. The PVA may be mixed with sodium trimetaphosphate solution prior to printing followed by polymer cross-linking during a drying process, in at least some cases. The first side of the mesh may be 3D printed onto the mesh as decellularized tissue matrix. In other cases, a mesh comprising a network of structures may be coated with PVA-P. After PVA-P crosslinking and 3D-printing of the Biomesh, it will be surgically implanted on the weakened or opened site.

In this disclosure, a multifunctional Biomesh with in situ phosphate crosslinked polyvinyl alcohol polymer (PVA-P) composite may be fabricated by 3D bioprinting (FIG. 2). The Biomesh possesses an optimal tensile strength to provide the required mechanical support to a hernia site, as an example. In particular embodiments, decellularized tissue gel is printed on the parietal side of the Biomesh to promote rapid tissue integration and healing [8]. In specific aspects, this Biomesh is novel at least because the PVA-P polymer is noninflammatory, prevents adhesion formation on the visceral side, while the parietal side containing decellularized gel matrix stimulates abdominal muscle tissue regeneration and healing of the hernia. The Biomesh in particular cases is programmed to slowly degrade after about 24 months of its implantation. The Biomesh prevents postoperative adhesion formation and stimulates rapid tissue integration. The Biomesh after surgical implantation enhances tissue healing (such as the abdominal wall) and reduces postoperative morbidity. The Biomesh possesses good suture holding capacity, will not tear under extreme mechanical stress (such as occurs while coughing or jumping), induces a desired host response, integrates with surrounding tissues and demonstrates mechanical properties sufficient for abdominal wall stabilization.

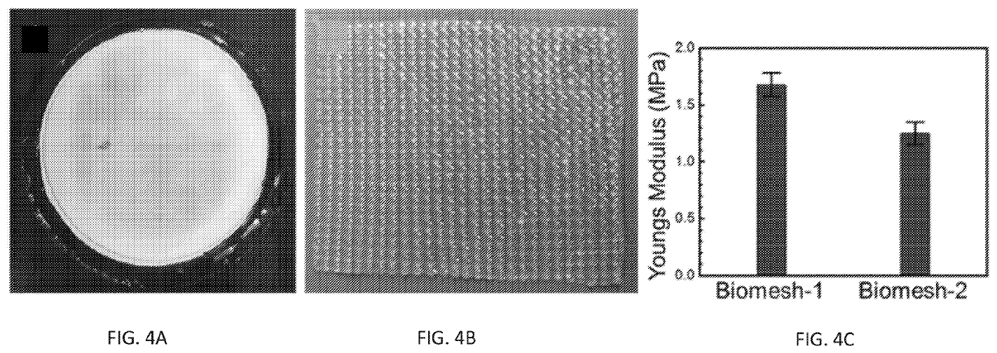

The Biomesh in specific embodiments was fabricated by 3D bioprinting using PVA-P composite. The PVA-P composite was synthesized by crosslinking of PVA with sodium trimetaphosphate (STMP) (FIG. 3). The crosslinking density of PVA-P composite can be optimized to provide the required tensile strength and mechanical stability to the Biomesh. In initial studies, the inventors have fabricated various Biomesh of tensile strengths 1.68 MPa and 1.25 MPa (FIG. 4C). Although the tensile strength values are very high, they are still highly flexible. In particular embodiments, the disclosure encompasses various Biomesh having a series of tensile strengths ranging from 23 kPa to 500 kPa. A deterioration of the tensile strength of the mesh could cause the Biomesh to shrink or curl and potentially lead to hernia recurrence or a poor functional result. Hence, the PVA-P composition in the Biomesh will be optimized to possess a tensile strength of >23 kPa to provide effective and functional support to the hernia site.

The maximum intra-abdominal pressure developed in a healthy individual is ˜20 kPa while jumping or coughing and the elastic modulus of the abdominal wall is ˜80 kPa [2, 3]. The mechanical strength of the Biomesh may be optimized to provide the required ability to withstand the maximum intra-abdominal pressure repeatedly without any deterioration of its mechanical strength for several months. Rapid deterioration of the mechanical strength of the mesh could potentially lead to hernia recurrence or a poor functional result. The Biomesh thickness and PVA-P crosslinking density may be optimized to possess an elastic modulus ≥80 kPa to provide effective and long-term functional support to the hernia site.

The inflammatory response to implanted mesh plays a crucial role in hernia repair surgery outcomes. A dysregulated inflammatory response can stimulate visceral adhesion formation, fibrosis, and disrupt wound healing process, thus leading to adverse complications requiring a revision surgery [4]. The PVA-P composite is noninflammatory and prevents the formation of visceral adhesions, seroma, and hematoma around the Biomesh upon surgical implantation.

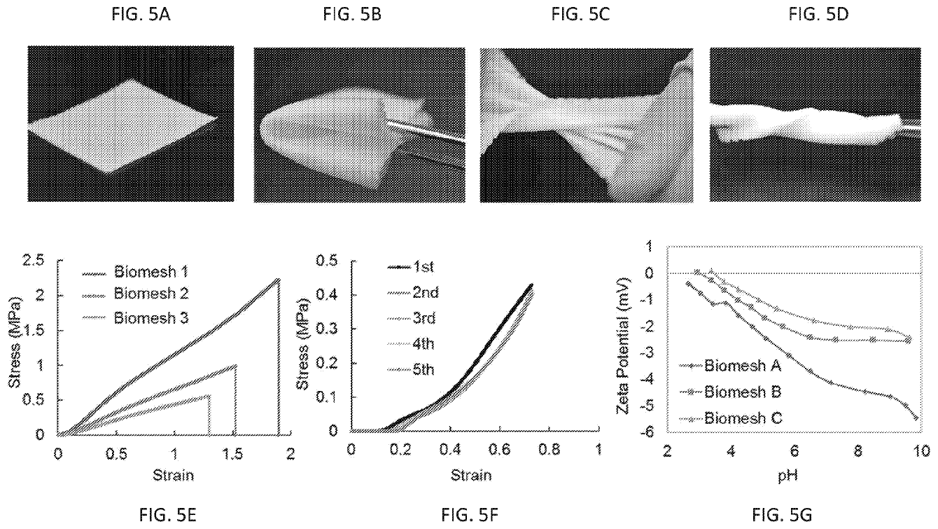

The PVA-P composite possesses an intrinsic net negative charge on its surface and behaves as an efficient adhesion barrier. Immunogenic and inflammatory responses to surgical materials strongly depend on surface properties such as surface charge, surface chemistry, and roughness of the implanted biomedical prosthetic, which influence the adsorption of proteins, growth factors and cellular elements migrating or growing into the area of repair [32]. The surface charge of the Biomesh plays an important role in modulating the inflammatory response after its implantation [33]. Zeta potential measurement of the Biomesh yields insights into the effect of surface charge on the in vivo immunogenic and inflammatory responses. Since the phosphate crosslinking groups are negatively charged, the surface charge of the Biomesh can be programmed by adjusting the phosphate crosslinking density. At pH 7, the average zeta potential values of highly crosslinked bioscaffold is −4.2 mV, medium crosslinked bioscaffold is −2.5 mV, and lowly crosslinked bioscaffold is −1.9 mV (FIG. 5G). Taken together, in specific embodiments, the mechanical properties, suture holding capacity, and the zeta potential of the Biomesh may be optimized by adjusting the phosphate crosslinking density and 3D-fabrication parameters.

In specific embodiments, the Biomesh is programmed to degrade after a certain period of time, such as 24 months, during which the hernia will regain strength and heal completely. In some cases, the Biomesh will degrade within 12-36 months, 12-30 months, 12-24 months, 12-18 months, 18-36 months, 18-24 months, or 24-36 months following implantation.

In particular embodiments of production of the Biomesh, the printer deposits a pre-polymer solution that polymerizes during the printing process. The phosphate crosslinking STMP is mixed with the polymer solution prior to printing. During the drying process, the polymer cross-linking occurs. With respect to the degree of cross linking being related to the rate of degradation, the degree of crosslinking is the only parameter that is a function of controlling the Biomesh degradation.

In particular cases, the Biomesh is not produced by 3D printing. In specific cases, the Biomesh is manufactured as a cast film.

### III. Methods of Use of the Compositions

In certain embodiments of the disclosure, a multifunctional Biomesh is utilized for repair of a weakness or opening of any kind in an individual. The weakness or opening may be caused by natural causes or may be caused upon intervention by man, such as with a medical procedure, including a surgical procedure, for example.

The disclosure provides a variety of embodiments of a Biomesh for any weakening or opening of a tissue of any kind in a mammal. In specific embodiments, the weakening or opening is in a muscular wall of a mammal. The Biomesh particularly is utilized for surgical repairs that can prevent visceral adhesion formation; stimulate rapid tissue integration; and prevent recurrence and prevent revision surgery.

The Biomesh described herein encompasses PVA-P membranes or meshes as an adhesion barrier in any kind of medical procedure, including hernia repair, cardiothoracic surgeries, tendon repair, pelvic floor repair, and breast implant surgery, for example.

In particular cases, an individual in need of repair of a weakening or opening in a tissue, such as muscle tissue, is provided at the time of repair a composition comprising PVA-P. The composition may be obtained commercially or may be produced at the time of the procedure or just prior to the time of the procedure to repair the weakening or opening. In cases wherein the Biomesh is produced at or just prior to the procedure, the size and/or content is determined in advance to tailor the composition to the site in need. For example, the size of the Biomesh is produced to fit the weakening or opening so that the weakening or opening is respectively adequately covered without unnecessary excess. The Biomesh may be selected or produced specifically to comprise a particular micropattern on the side of the Biomesh that faces towards the wall.

In particular cases, the tensile strength of the Biomesh is customized to be effective for a particular weakening or opening in the individual. The Biomesh may be produced to have a tensile strength greater than 23 kPa. The required strength of the Biomesh may be determined ahead of the medical procedure and the Biomesh may be produced to have a particular tensile strength based upon the crosslinking produced upon 3D printing.

### EXAMPLES

The following examples are included to demonstrate preferred embodiments of the invention. It should be appreciated by those of skill in the art that the techniques disclosed in the examples that follow represent techniques discovered by the inventor to function well in the practice of the invention, and thus can be considered to constitute preferred modes for its practice. However, those of skill in the art should, in light of the present disclosure, appreciate that many changes can be made in the specific embodiments which are disclosed and still obtain a like or similar result without departing from the spirit and scope of the invention.

### Example 1

### Biomesh Preparation

This study was designed to evaluate the in vivo efficacy of the Biomesh in preventing visceral adhesions formation and its inflammation modulatory properties by the sequestration of proinflammatory cytokines. The Biomesh was fabricated by 3D bioprinting using phosphate crosslinked polyvinyl alcohol polymer. The mechanical properties of the Biomesh were studied by rheometry. The surface charge densities were measured by zeta potential measurements.

Synthesis of PVA-P polymer: A bioscaffold with in situ phosphate crosslinked poly (vinyl alcohol) polymer (PVA-P) was synthesized by the reaction of PVA with sodium trimetaphosphate (STMP) at room temperature (FIG. 3). Ten grams of PVA (Sigma-Aldrich Inc., MW 85,000-124,000; 87-89% hydrolyzed) was dissolved in 100 mL of distilled water and stirred until complete dissolution. Once PVA was completely dissolved, 10 mL of PVA solution was transferred into a glass vial and 7.5 mL of 15% (w/w in water) sodium trimetaphosphate (Sigma-Aldrich) solution was added to the vial. The PVA and STMP solution was constantly stirred to obtain a homogeneous mixture. Then 3 mL of sodium hydroxide (30%; w/w in water) was added to PVA-STMP solution drop wise under constant stirring. The solution was let stand for 30 minutes before using for the Biomesh fabrication.

Biomesh fabrication: The Biomesh was fabricated using a 3D-bioprinter (3DDiscovery™; regenHU, Switzerland). The design of the Biomesh was created by BioCad™ (regenHU, Switzerland, FIGS. 2B-2D). The Biomesh was fabricated by layer-by-layer printing of PVA-P and STMP solutions with in situ cross-linking. Briefly, a 5-cm³ cartridge filled with PVA-STMP solution was attached with a 27 G needle and placed in a print head. The Biomesh was printed in contact mode, 0.01 mm above the surface as the PVA-STMP solution was directly dispensed layer-by-layer on a glass plate with a pressure of 0.5 MPa and a collector velocity of 10 mm/s until the desired dimension was achieved. After printing, the Biomesh was dried overnight and rinsed thoroughly with ultra-pure water.

Mechanical testing and suture holding capacity of Biomesh: All mechanical testing of the Biomesh was performed using the ARES-G2 rheometer (TA Instruments; New Castle, Del.). To determine the elastic modulus, the Biomesh was cut into a rectangular shape (length 30 mm×width 10 mm×thickness 0.3-0.5 mm). The exact dimensions of the Biomesh were measured with a caliper prior to tensile testing performed at a speed of 0.1 mm/s. The stress-strain curve was obtained and used to calculate the elastic modulus. In another study, the top and bottom of the Biomesh was sutured with a PROLENE suture to evaluate its suture holding capacity. The sutures were inserted into the tension clamps and tensile testing was performed at a speed of 0.1 mm/s until 70% strain was reached up to five cycles.

Results—mechanical testing and suture holding capacity: In initial studies, Biomesh with tensile strengths 1.68 MPa and 1.25 MPa were fabricated (FIG. 4C). In additional studies, three Biomesh of elastic moduli ˜400, 700, and 1000 kPa were fabricated by additive 3D-bioprinting (FIG. 5E). Biomesh could also retained sutures for up to 100 kPa without failure, which is ˜5 times that of the maximum intra-abdominal pressure (20 kPa) developed in healthy individuals (FIG. 5F).

Zeta potential measurements: The surface zeta potential of the Biomesh was measure using the SurPASS™ electrokinetic analyzer (Anton Paar. Graz, Austria) equipped with an adjustable gap cell. The zeta potential of the Biomesh is determined by measuring the streaming current or streaming potential. Using a clamping cell, the Biomesh was loaded on one side of the electrolyte channel. The surface zeta potential was determined as a function of pH in KCl electrolyte solution. For the measurements, 5 mM KCl solution was used as the source of electrolyte and purged with nitrogen throughout the experiment. The pH of electrolyte solution was adjusted by the addition of 25 mM HCl or 25 mM NaOH solutions through the instrument's automatic titration unit. In between measurements, the sample was rinsed twice with the electrolyte solution. Separate couples of samples were used for the acidic and basic titrations in order to avoid artifacts due to surface reactions during the measurement. Four measurements were carried out for each pH point. The zeta potential, ζ, is calculated according to the following Fairbrother-Mastin equation for streaming current measurements:

$$ζ = \frac{d U}{d P} \cdot \frac{η}{ɛ \cdot ɛ o} \cdot k$$

wherein: dU/dP: Slope of streaming potential vs. differential pressure; η: Electrolyte viscosity εₒ: Permittivity; ε: Dielectric coefficient of electrolyte; k: Conductivity.

Results—zeta potential measurement: Three Biomesh were fabricated with increasing phosphate crosslinking densities to program the surface charges of the Biomesh. At pH 7, the average zeta potential values of a highly crosslinked Biomesh is −4.2 mV, a medium crosslinked Biomesh is −2.5 mV, and a lowly crosslinked Biomesh is −1.9 mV (FIG. 5G).

### Example 2

### Biomesh Acts as an Inflammation Trap

In vitro bead-based assay for cytokine binding study: An in vitro bead-based assay was performed to test Biomesh ability to capture the inflammatory cytokines TNF-α, IL-1α, IL-6 and VEGF as depicted in FIG. 6A [34, 35].

Preparation of cytokine solution: To prepare the cytokine solution, mouse cytokine quality control-2 (MXM6070-2) from Luminex assay kit (MCYTOMAG-70K, EMD Millipore) was prepared as per the given instructions. In brief, the contents in the vial was reconstituted with 250 μL MilliQ water and thoroughly mixed. The vial was allowed to sit for 5-10 minutes at room temperature and then transferred to a 15 mL polypropylene tube. The cytokine solution was then constituted to 10 mL (1:40 dilution) using MilliQ water.

Preparation of antibody beads: Mouse Cytokine/Chemokine Antibody-Immobilized Magnetic Beads for IL-1β(MIL1B-MAG), IL-6 (MCYIL6-MAG), VEGF (MVEGF-MAG), TNFα(MCYTNFA-MAG), and MIP-1α(MMIP1A-MAG) from Luminex assay kit (EMD Millipore) were used for the study. The antibody beads were prepared as per the instructions given in the kit. In brief, each antibody-bead vial was sonicated for 30 seconds followed by vortex for 1 minute. Using the Assay buffer provided in the kit, 60 μL of each antibody bead solution was diluted to bring the final volume to 3.0 mL and mixed well.

In vitro cytokine binding assay: The 3D printed Biomesh was cut into small pieces (0.5 cm×0.5 cm), placed one in each well of a 24-well plate and allowed to hydrate in 1 mL of sterile 1× PBS overnight. After removing the PBS from each well, Biomesh pieces were washed three times with MilliQ water. From the prepared cytokine solution (IL-1β, IL-6, VEGF, TNFα, and MIP-1α), 300 μL was added to each well containing a small Biomesh piece except to the control well. In the control well, 300 μL of MilliQ water was added. The 24-well plate was then covered and incubated for 72 hours at 37° C. in an orbital shaker to allow the binding of cytokines on to the mesh. After incubation, the cytokine solution was removed, and each well was rinsed three times with MilliQ water. Then 300 μL of prepared antibody-bead solutions were added to each well, covered with aluminum foil and incubated for 24 hours in dark. After incubation, the antibody solution was removed, and wells were rinsed three times in MilliQ water. The Biomesh were carefully removed from the wells, placed on a clean slide and covered with a glass cover slip. The prepared slides were allowed to dry overnight and imaged and analyzed using 10× objective of Nikon Eclipse Ti inverted microscope equipped with differential interference contrast (DIC) imaging and fluorescence (widefield) microscopy (Far Red—640 filter). Each experiment was done in triplicates (n=3).

Results—in vitro cytokine binding: This study revealed that the pro-inflammatory cytokines TNF-α, IL-1β, and IL-6 were bound to the Biomesh surface. FIGS. 6B-6G present overlays of the brightfield and fluorescence images present the cytokine bound Biomesh surface. For clarity, the fluorescence images were presented next to the overlay images. The antibody beads did not bind to the Biomesh in the absence of cytokines on the Biomesh surface (FIG. 6B). The TNF-α antibody coupled beads selectively bound to the TNF-α adsorbed on the Biomesh surface (FIG. 6C). Similarly, IL-1α, IL-6, and MIP-1α antibody coupled beads also selectively bound to the corresponding IL-1α, IL-6, MIP-1α VEGF adsorbed on the Biomesh surface (FIGS. 6D-6G). This study confirmed the intrinsic ability of Biomesh to capture inflammatory cytokines.

### Example 3

### Biomesh Prevents Adhesion Formation

Study design: Sample size justification for our study was performed using power analysis. We used n=6 animals per group for the in vivo experiments. This study aimed to evaluate the Biomesh as a final treatment for the repair of abdominal wall hernias while proving its capability to prevent the development of adhesions, its mechanical strength to withstand abdominal wall forces and its ability to reduce hernia recurrence rates. In vivo studies included 3 groups and 2 different time points. All study subjects underwent ventral hernia creation and repair surgery. In the first group, hernias were repaired using commercially available polypropylene mesh. In the second group, Biomeshs were used to repair hernias, while PVA-P coated polypropylene meshes were used for the third group. End Points (2 and 4 weeks) were chosen to evaluate early adhesion formation and mesh contraction. Samples were collected for molecular and histological analysis.

Ventral hernia rat model creation and repair: For the purpose of our study, all animals were maintained and used in accordance with an IACUC approved animal protocol by Baylor College of Medicine and the American Association for Laboratory Animal Science (AALAS). Sprague Dawley rats (300 grams; Charles River Laboratories, Houston, Tex., USA) were used for in vivo evaluations. Pre- and Post-operative, weight-based analgesia was given to all study subjects. Anesthesia was induced and maintained by using inhaled isoflurane gas. After preparation of the surgical site, in supine position and observing sterile technique in order to minimize the contamination of the surgery site, all animals underwent ventral hernia creation and repair surgery. A 3 cm skin flap was raised through the avascular pre-fascial plane. After performing skin dissection, a 2 cm midline laparotomy incision was then made using the Linea Alba as the surgical mark. After assuring hemostasis of the surgical site, an intraperitoneal underlay repair was performed. The mesh was placed between the contents of the abdominal cavity and the abdominal wall, using a 0.5 cm overlap of each mesh with the respective fascial edge of the abdominal tissue. The mesh was anchored at multiple equidistant points using 4-0 PROLENE sutures. Wound clips and skin adhesive were used for skin closure.

Tissue harvesting: Once the study subjects reached their respective endpoint, animals were humanely euthanized following the Animal Welfare Act guidelines. After skin dissection, an abdominal muscle wall flap was created in order to expose the hernia repair site and to evaluate the presence of adhesions. Samples for histology were taken and fixed in formalin prior to processing, paraffin embedding, and sectioned using a microtome for H&E staining. Molecular analysis samples were stored in RNAlater® Stabilization Solution (Ambion™) and stored at room temperature and processed within 24 h of sample collection.

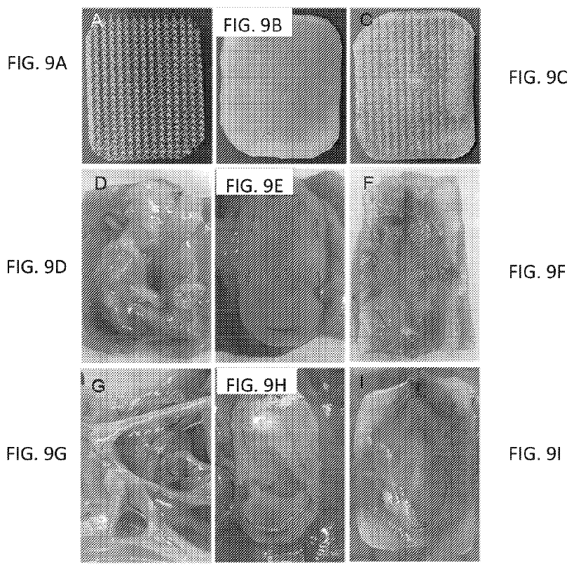

Results—adhesion formation: To evaluate the efficacy of the Biomesh in preventing adhesion formation, three types of meshes were tested in ventral hernia rat model: (1) Polypropylene (PROLENE®) mesh as a control (FIG. 9A), since it is in extensive clinical use for surgical hernia repair and known to cause serious adhesion formation [17], (2) Biomesh (FIG. 9B), and (3) PVA-P coated PROLENE® mesh to demonstrate the efficacy of PVA-P polymer in preventing adhesion formation (FIG. 9C). For this study, rats were divided into three groups (n=4) and the first group was implanted with PROLENE® mesh, the second group was implanted with Biomesh, and the third group was implanted with PVA-P coated PROLENE® mesh. The adhesion formation and abdominal wall healing was evaluated at 2 and 4-week timepoints.

The PROLENE® mesh triggered an extreme level of adhesion formation spread all over the mesh at 2- and 4-week time points (FIGS. 9D and 9G). After 2 weeks of implantation, the PROLENE® mesh was completely covered with adhesions, the sutures and mesh were not visible (FIG. 9D). After 4 weeks, the PROLENE® mesh, in addition to complete coverage with firm adhesions the underlying omentum, liver and bowel were also attached to it (FIG. 9G). In comparison, the Biomesh is highly effective in preventing adhesion formation. The Biomesh elicited no adhesion formation even after 4 weeks (FIGS. 9E and 9H). The PVA-P coated PROLENE® mesh was also as effective as Biomesh in preventing adhesion formation and the mesh and the sutures are clearly visible even after 4 weeks (FIGS. 9F and 9I).

Histology: For H&E staining, the paraffin embedded tissue samples were sectioned at 10 μm using an Accu-Cut SRM microtome (Sakura, Japan) and collected on a glass slide. The tissue section-mounted glass slides were stained with hematoxylin for 4 min and eosin for 1 min followed by dehydration and clearing in ethanol and xylene. The glass slides were then mounted using Permount Mounting medium (Thermo Fisher Scientific, USA) and sealed with a glass coverslip. The sections were imaged and analyzed using Nikon eclipse TE2000-U microscope.

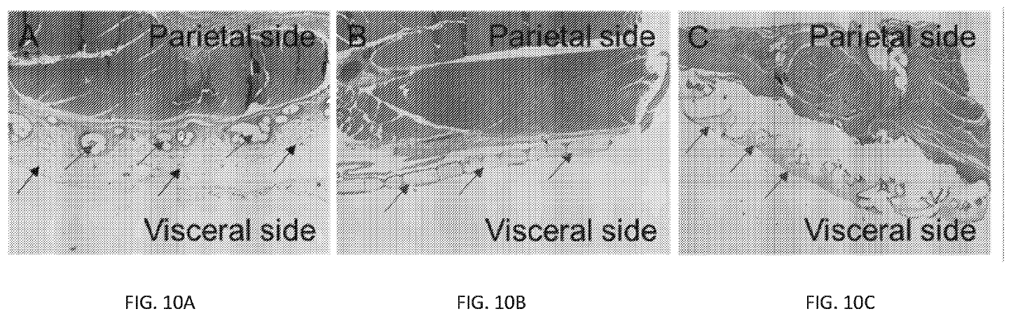

Results—histology: To evaluate the efficacy of the Biomesh in preventing adhesion formation, histological analysis of the implanted mesh sections was performed by H&E staining. As can be seen from the FIG. 10, PROLENE® mesh developed a thick layer of peritoneum adhering to it (FIG. 10A), while the Biomesh and the PVA-P coated PROLENE® mesh did not develop any kind of visceral adhesions at all (FIGS. 10B and 10C), thus confirming the potential of Biomesh and the PVA-P in preventing visceral adhesion formation after surgical hernia repair.

Macroscopic adhesions grading: The macroscopic grading of postoperative adhesions in rats was based on the assessment of the following three characteristics: Extent, Type, and Tenacity. Graded on a scale of 0 to 4, the extent of the adhesions was evaluated. In this scale, 0 meant no adhesions present while 4 represented full coverage of the surgical mesh. On a scale of 0 to 3, the adhesion type was evaluated as well. Physical characteristics of the adhesions such as the density of the adhered tissues and the presence of blood vessels in said tissues were scored. As in the previous scale, 0 meant no adhesions present on the surgical mesh, while on the other end of the scale, 3 meant dense adhesions with blood vessels present. The force required to detach the adhered tissues or in some cases, the use of surgical tools to achieve this detachment, was evaluated under the 0 to 3 tenacity score. Just as with the previous two characteristics evaluated, the absence of adhesions was scored 0, and the use of sharp dissection was scored 3.

Extent: Assesses total mesh area covered by adhesions, on a scale from 0 to 4 (0=absence of adhesions; 1=0-25% of the mesh is covered with adhesions; 2=25-50% of the mesh is covered with adhesions; 3=50-75% of the mesh is covered with adhesions; 4=75-100% of the mesh is covered with adhesions).

Tenacity: Assesses the force required to detach the adhered tissues from the mesh or if the use of surgical tools was needed to achieve this detachment (0=absence of adhesions; 1 =adhesions easily fall apart; 2=adhesions need to be pulled to separate; 3=adhesions require sharp dissection to separate from the mesh).

Type: Assesses the density of the adhered tissues, as well as the presence of blood vessels in said tissues on a scale of 0 to 3 (0=no adhesions; 1=filmy; 2=dense; 3=capillaries present).

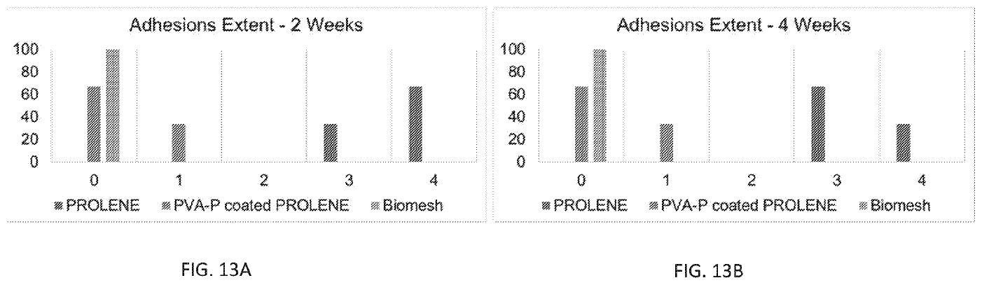

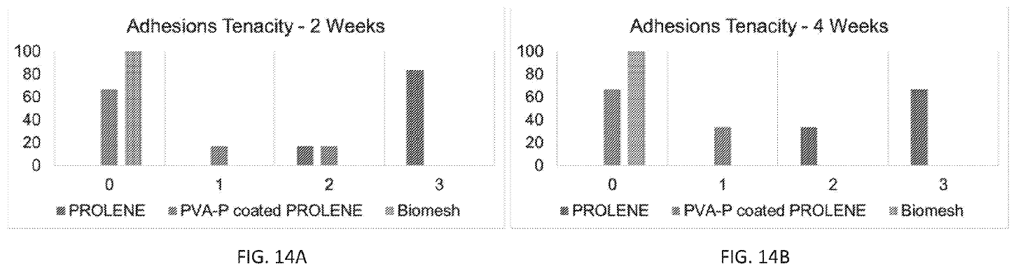

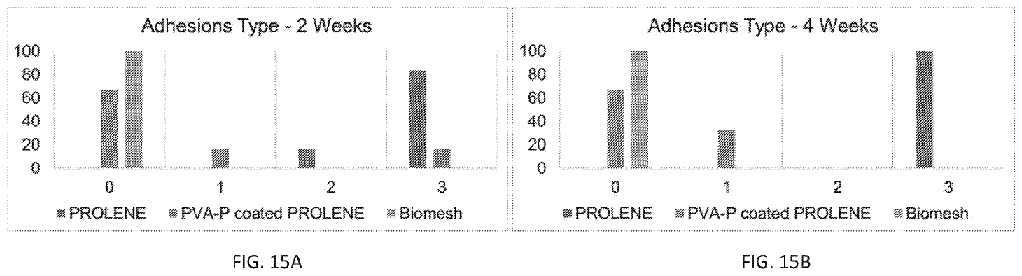

Results—macroscopic adhesions grading: Clinical grading measured the extent (FIG. 13), tenacity (FIG. 14), and type (FIG. 15) of postsurgical adhesions formation at 2-week (FIGS. 13-15A) and 4-week (FIGS. 13-15B) timepoints. Results showed that Biomesh and PVA-P coated PROLENE® mesh did not form adhesions in comparison to the PROLENE mesh.example 4

### Example 4

### Biomesh is Noninflammatory

Quantification of inflammatory cytokines by quantitative Polymerase Chain Reaction: Total RNA from the samples were extracted using TRIzol reagent (Ambion, Carlsbad, Calif., USA.) and stored at −80° C. RNA was quantified, and quality was assessed using Nanodrop 2000 spectrophotometer (Thermo Fisher Scientific, USA). First-strand cDNA was synthesized from 1.0 μg of RNA with Ready-To-Go You-Prime-First-Strand Beads (GE Healthcare, Princeton, N.J., USA) and random hexamers (Applied Biosystems, Thermo Fisher Scientific, USA). Equal amounts of synthesized cDNA were then used to measure specific gene expression by qPCR using a TaqMan Fast Advanced Master Mix (Applied Biosystems, Thermo Fisher Scientific, USA) for specific primers—IL-1B (Rn00580432-m1), IL-6 (Rn01410330-m1), IL-8 (Rn02130551-s1), TNF-α (Rn01525859-g1), TGF-β(Rn00676060-m1), and VEGF (Rn01511602-m1) from Applied Biosystems on Quantstudio 5 Real-time PCR system (Applied Biosystems, Thermo Fisher Scientific, USA). The fold change value of relative gene expression was calculated using the ΔΔCT quantification method with GAPDH (Rn01775763-g1) as an internal reference.

Statistical Analysis of qPCR results: Data in figures are presented as mean±SD. Statistical significance of comparison of mean values was assessed by unpaired T-Test (two tailed P-value) using GraphPad Prism 8.0 software (GraphPad Incorporation, San Diego, Calif.).

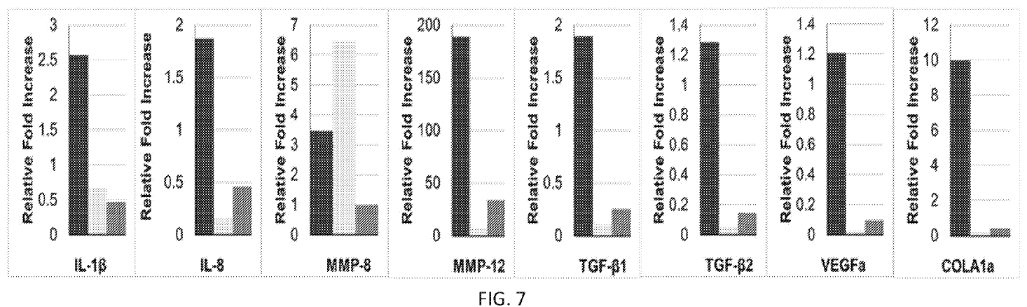

Results—inflammatory response: Inflammatory responses after the implantation of the Biomesh was analyzed by measuring the expression levels of proinflammatory cytokines IL-1β, IL-8, proinflammatory matrix metalloproteases MMP-8, MMP-12, proangiogenic VEGF-A, and profibrotic TGF-β1, TGF-β2, Col-1a and and compared with PROLENE® mesh by RT-PCR analysis. As can be seen from FIG. 7, the Biomesh was very effective in suppressing the expression levels of IL-1β, IL-8, MMP-8, MMP-12, VEGF-A, TGF-β1, TGF-β2, and COL-1a. This study revealed that the Biomesh is noninflammatory and did not elicit an immune reaction after the implantation of the Biomesh at 7-day timepoint in comparison to the PROLENE® mesh, which initiated a strong inflammatory response as evidenced at least by the pronounced expression levels of IL-1β, IL-8, MMP-8, MMP-12, VEGF-A, TGF-β1, TGF-β2, and COL-1a (FIG. 7).

Inflammatory responses after the implantation of the Biomesh were additionally analyzed by measuring the expression levels of IL-1β, IL-6, IL-8, TNF-α, MMP-8, TGF-β1, and VEGF and compared with PROLENE® mesh by qPCR analysis. As can be seen from FIG. 8, the Biomesh was very effective in suppressing the expression levels of IL-1β, IL-6, IL-8, TNF-α, MMP-8, TGF-β1, and VEGF. This study further supports that the Biomesh is noninflammatory and did not elicit an immune reaction after the implantation of the Biomesh at 7-day timepoint in comparison to the PROLENE® mesh, which initiated a strong inflammatory response as evidenced at least by the pronounced expression levels of IL-1β, IL-8, MMP-8, TNF-α, MMP-12. TGF-β1, and VEGF (FIG. 8). On the other hand, PROLENE® mesh initiated a strong inflammatory response and caused severe adhesions formations.

### Example 5

### Biomesh Stimulates Abdominal Wall Healing Process

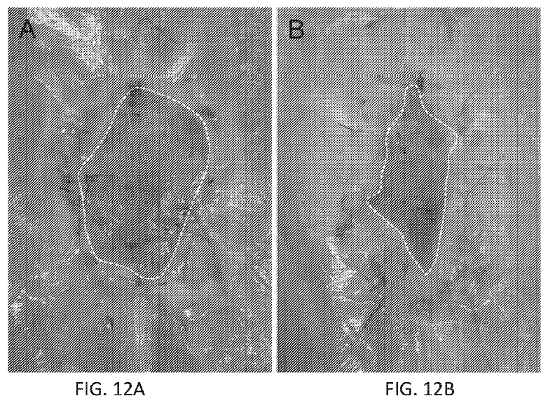

Biomesh stimulates abdominal wall healing in ventral hernia rat model after surgical hernia repair (FIG. 12). In the case of hernia repaired with the Biomesh, the wound is rapidly healing at the 2-week time point (FIG. 12A) and approaching the wound closure at the 4-week timepoint (FIG. 12B). The margins of the Biomesh and the sutures are clearly visible.

### Example 6

### Biomesh Surface Chemistry and Charge

### Affects Adhesions Formation

The effect of the Biomesh surface charge on inflammation and adhesions formation was examined in a rat ventral hernia model. The in vivo efficacy of the Biomesh in preventing visceral adhesions formation and its inflammation modulatory properties were studied in a rat ventral hernia model and compared with present standard of care polypropylene mesh.

Surface Charge-Modified Biomesh fabrication: To evaluate the effect of surface charge on adhesion formation, Biomesh of three different surface charges were fabricated: (1) Biomesh with a negatively charged surface, (2) Biomesh with a neutral surface, and (3) Biomesh with a positively charged surface. As prepared by our layer-by-layer 3D-printing method, Biomesh has a net negative surface charge because of the phosphate groups. A neutral Biomesh was prepared by soaking the material in HCl (1M, 30 min) to convert the negatively charged phosphate groups to phosphoric acid groups. Positively charged Biomesh was prepared by soaking it in polylysine hydrobromide solution (1 mg/ml, 12h) to form a polylysine monolayer on its surface.

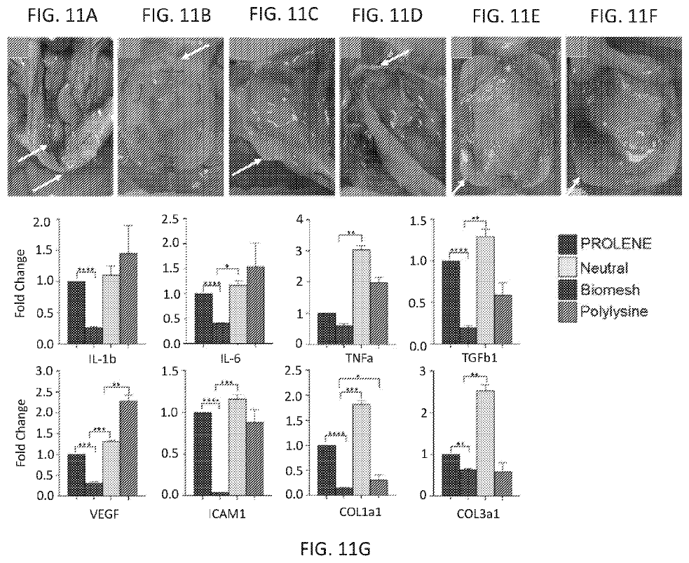

Results—adhesions formation: An important point to note is that a change in the surface charge also altered the surface chemistry. To evaluate the effect of surface charge on postsurgical adhesion formation, these Biomesh were implanted in the rat ventral hernia model (FIGS. 11B-11D) and compared with PROLENE mesh (FIG. 11A). The negatively charged Biomesh did not elicit any adhesion formation on its surface and the sutures are clearly visible (FIG. 11B). In the case of neutral Biomesh, extensive peritoneal adhesions formation was observed, possibly because of the acid groups on the surface (FIG. 11C). In the case of positively charged polylysine coated Biomesh, again extensive fibrotic adhesions formation was observed (FIG. 11D). The effect of fibronectin coated Biomesh and gelatin coated Biomesh on adhesion formation was studied. Fibronectin is a positively charged ECM glycoprotein that plays a key role in early wound healing and tissue repair. Fibronectin and gelatin coated Biomesh also triggered adhesions formation within 2 weeks (FIGS. 11E and 11F).

Results—inflammatory response: To evaluate the effect of surface charge on inflammation and visceral adhesion formation at the molecular level, the tissue surrounding the implant site was analyzed for the expression levels of the IL-1β, IL-6, TNF-α, TGF-β1, VEGF, ICAM 1, COL1a1, and COL3a1 and compared with the PROLENE mesh by qPCR analysis. As can be seen from FIG. 11G, expression levels of proinflammatory cytokines in the cases of neutral and positively charged Biomesh were as high as those observed for PROLENE mesh. On the other hand, the negatively charged Biomesh elicited negligible inflammatory response and the expression levels of proinflammatory cytokines were minimal.

Taken together, the positively charged and neutral Biomesh (FIGS. 11C-11F) triggered extensive adhesions formation that were as significant as seen with the PROLENE mesh (FIG. 11A). This study clearly demonstrates the importance of negative charge on the Biomesh surface in preventing postsurgical visceral adhesion formation. A mild alteration of the surface charge on the Biomesh by the adsorption of a monolayer of positively charged polylysine or fibronectin produced significantly higher levels of inflammatory cytokine expression resulting in the formation of vascularized adhesions that covered the Biomesh and connected the implanted material to the liver and intestine. These results confirm the important considerations of surface chemistry and surface charge in Biomesh design.

In summary, these experimental results have demonstrated: (i) Biomesh fabrication; (ii) the ability of Biomesh to capture inflammatory cytokines on the Biomesh surface, (iii) noninflammatory nature of the Biomesh, (iv) the ability of the Biomesh to prevent visceral adhesion formation in the long-term compared to the PROLENE® mesh.

### REFERENCES

All patents and publications mentioned in this specification are indicative of the level of those skilled in the art to which the invention pertains. All patents and publications herein are incorporated by reference to the same extent as if each individual publication was specifically and individually indicated to be incorporated by reference in their entirety.
1. BK Poulose, J Shelton, S Phillips, D Moore, W Nealon, D Penson, W Beck, MD Holzman. Epidemiology and cost of ventral hernia repair: making the case for hernia research. Hernia 2012, 16:179-83.
2. PP Pott, MLR Schwarz, R Gundling, K Nowak, P Hohenberger, ED Roessner. Mechanical properties of mesh materials used for hernia repair and soft tissue augmentation. PLoS ONE 2012 7(10): e46978. doi:10.1371/journal.pone.0046978
3. CR Deeken, CP Lake. Mechanical properties of the abdominal wall and biomaterials utilized for hernia repair. J Mech Behav Biomed Mater. 2017, 74:411-427.
4. K Baylón, P Rodríguez-Camarillo, A Elías-Zuniga, JA Díaz-Elizondo, R Gilkerson, K Lozano. Past, present and future of surgical meshes: A review. Membranes 2017, 7: 47, doi:10.3390/membranes7030047
5. JL Holihan, Z Alawadi, RG Martindale, JS Roth, CJ Wray, TC Ko, LS Kao, MK Liang. Adverse events after ventral hernia repair: the vicious cycle of complications J Am Coll Surg. 2015, 221:478-85.
6. T Liakakos, N Thomakos, PM Fine, C Dervenis, RL Young. Peritoneal adhesions: Etiology, pathophysiology, and clinical significance. Recent advances in prevention and management. Dig Surg. 2001, 18:260-273.
7. H Ellis, BJ Moran, JN Thompson, MC Parker, MS Wilson, D Menzies, A McGuire, AM Lower, RJS Hawthorn, F O'Brien, S Buchan, AM Crowe. Adhesion-related hospital readmissions after abdominal and pelvic surgery: A retrospective cohort study. Lancet 1999, 353:1476-1480.
8. S Lanouar, R Aid-Launais, A Oliveira, L Bidault, B Closs, M-N Labour, D Letourneur. Effect of cross-linking on the physicochemical and in vitro properties of pullulan/dextran microbeads. J Mater Sci Mater Med. 2018; 29:77.
9. S Lack, V Dulong, L Picton, D Le Cerf, E Condamine. High-resolution nuclear magnetic resonance spectroscopy studies of polysaccharides crosslinked by sodium trimetaphosphate: a proposal for the reaction mechanism. Carbohydrate Res. 2007, 342:943-953.
10. M Chaouat, C Le Visage, WE Baille, B Escoubet, F Chaubet, MA Mateescu, D Letourneur. A novel cross-linked poly(vinyl alcohol) (PVA) for vascular grafts. Adv Funct Mater. 2008, 18:2855-2861.
11. JC Fricain, R Aid, S Lanouar, DB Maurel, D Le Nihouannen, S Delmond, D Letourneur, JA Vilamitjana, S Catros. In-vitro and in-vivo design and validation of an injectable polysaccharide-hydroxyapatite composite material for sinus floor augmentation. Dental Mater 2018, 34:1024-1035.
12. CN Brown, JG Finch. Which mesh for hernia repair? Ann R Coll Surg Engl. 2010, 92:272-278. doi 10.1308/003588410X12664192076296
13. CR Deeken, BD Matthews. Characterization of the mechanical strength, resorption properties, and histologic characteristics of a fully absorbable material (Poly-4 hydroxybutyrate—PHASIX Mesh) in a porcine model of hernia repair. ISRN Surgery 2013 http://dx.doi.org/10.1155/2013/238067
14. S Kalaba, E Gerhard, JS Winder, EM Pauli, RS Haluck, J Yang. Design strategies and applications of biomaterials and devices for hernia repair. Bioactive Mater. 2016, 2-17
15. Y Bilsel, I Abci. The search for ideal hernia repair; mesh materials and types. Int J Surgery. 2012, 10: 317e321
16. F Heymann, KT vo Trotha, C Preisinger, P Lynen-Jansen, AA Roeth, M Geiger, LJ Geisler, AK Frank, J Conze, T Luedde, C Trautwein, M Binnebosel, UP Neumann, F Tacke. Polypropylene mesh implantation for hernia repair causes myeloid cell-driven persistent inflammation. JCI Insight. 2019 Jan 24;4(2). pii: 123862. doi: 10.1172/jci.insight.123862.
17. ME Falagas, SK Kasiakou. Mesh-related infections after hernia repair surgery. Clin Microbial Infect. 2005, 11:3-8.
18. SR Markar, A Karthikesalingam, F Alam, TY Tang, SR Walsh, U Sadat. Partially or completely absorbable versus nonabsorbable mesh repair for inguinal hernia: A systematic review and meta-analysis. Surg Laparosc Endosc Percutan Tech. 2010, 20:213-219.
19. S Oberg, K Andersen, J Rosenberg. Absorbable meshes in inguinal hernia surgery: A systematic review and meta-analysis. Surg Innov. 2017, 24:289-298.
20. S Elango, S Perumalsamy, K Ramachandran, K Vadodaria. Mesh materials and hernia repair. BioMedicine 2017, 7:14-23.
21. JM Becker, MT Dayton, VW Fazio, DE Beck, SJ Stryker, SD Wexner, BG Wolff, PL Roberts, LE Smith, SA Sweeney, M Moore, J Rosenberg. Prevention of postoperative abdominal adhesions by a sodium hyaluronate-based bioresorbable membrane: a prospective, randomized, double-blind multicenter study. J Am Coll Surg. 1996, 183: 297-306.
22. L Shapiro, J-L Holste, T Muench, G diZerega. Rapid reperitonealization and wound healing in a preclinical model of abdominal trauma repair with a composite mesh. Int J Surgery 2015, 22: 86e91
23. MHF Schreinemacher, PJ Emans, MJJ Gijbels, J-WM Greve, GL Beets, ND Bouvy. Degradation of mesh coatings and intraperitoneal adhesion formation in an experimental model. Br J Surg. 2009, 96:305-313
24. B East, M Plencner, M Kralovic, M Rampichova, V Sovkova, K Vocetkova, M Otahal, Z Tonar, Y Kolinko, E Amler, J Hoch. A polypropylene mesh modified with poly-6-caprolactone nanofibers in hernia repair: large animal experiment. Int J Nanomedicine 2018,13:3129-3143 25. EB Deerenberg, J Verhelst, SER Hovius, JF Lange. Mesh expansion as the cause of bulging after abdominal wall hernia repair. Int J of Surg Case Reports 2016,28:200-203.
26. T Hufford, J-F Tremblay, MTM Sheikh, S Marecik, J Park, I Zamfirova, K Kochar. Local parastomal hernia repair with biological mesh is safe and effective. Am J Surg. 2018, 215:88-90.
27. C Shi, W Chen, Y Zhao, B Chen, Z Xiao, Z Wei, X Hou, J Tang, Z Wang, J Dai. Regeneration of full-thickness abdominal wall defects in rats using collagen scaffolds loaded with collagen-binding basic fibroblast growth factor. Biomaterials 2011, 32:753e759
28. JF FitzGerald, AS Kumar. Biologic versus synthetic mesh reinforcement: What are the pros and cons? Clin Colon Rectal Surg 2014, 27:140-148.
29. FE Primus, DM Young, JP Grenert, HW Harris. Silver microparticles plus fibrin tissue sealant prevents incisional hernias in rats. J Surge Res. 2018, 227: 130-136
30. F Pati, J Jang, D-H Ha, SW Kim, J-W Rhie, J-H shim, D-H Kim, D-W Cho. Printing three-dimensional tissue analogues with decellularized extracellular matrix bioink. Nat Commun. 2014 Jun 2; 5:3935. doi: 10.1038/ncomms4935.
31. G Voskerician, J Jin, SA Hunter, CP Williams, M White, MJ Rosen MJ. Human peritoneal membrane reduces the formation of intra-abdominal adhesions in ventral hernia repair: experimental study in a chronic hernia rat model. J. Surg. Res. 2009, 157: 108-114.
32. B. Kasemo. Biological surface science. Surf. Sci. 2002, 500:656-677. doi: 10.1016/50039-6028(01)01809-X
33. S. Ferraris, M. Cazzola, V. Peretti, B.Stella, S. Spriano. (2018) Zeta potential measurements on solid surfaces for in vitro biomaterials testing: surface charge, reactivity upon contact with fluids and protein absorption. Front. Bioeng. Biotechnol. 2018, 6:60. doi: 10.3389/fbioe.2018 0.00060
34. G. Acharya, C. L. Chang, D. D. Doorneweerd, E. Vlashi, W. A. Henne, L. C. Hartmann, P. S. Low, C. A. Savran. Immunomagnetic diffractometry for detection of diagnostic serum markers. J Am Chem Soc. 2007, 129, 51, 15824-15829.
35. G. Acharya, C. L. Chang, D. P. Holland, D. H. Thompson, C. A. Savran. Rapid Detection of S-Adenosyl Homocysteine Using Self-Assembled Optical Diffraction Gratings. Angew. Chem. Int. Ed. 2008, 47, 1051-1053.

Although the present disclosure and its advantages have been described in detail, it should be understood that various changes, substitutions and alterations can be made herein without departing from the spirit and scope of the design as defined by the appended claims. Moreover, the scope of the present application is not intended to be limited to the particular embodiments of the process, machine, manufacture, composition of matter, means, methods and steps described in the specification. As one of ordinary skill in the art will readily appreciate from the present disclosure, processes, machines, manufacture, compositions of matter, means, methods, or steps, presently existing or later to be developed that perform substantially the same function or achieve substantially the same result as the corresponding embodiments described herein may be utilized according to the present disclosure. Accordingly, the appended claims are intended to include within their scope such processes, machines, manufacture, compositions of matter, means, methods, or steps.

1. A composition comprising a mesh, said mesh comprising phosphate crosslinked poly(vinyl alcohol) polymer (PVA-P).

2. The composition of claim 1, wherein the mesh comprises a network of structures that are coated with PVA-P and/or the mesh comprises a network of structures made of PVA-P.

3. The composition of claim 1, wherein said mesh comprises a first side and a second side.

4. The composition of claim 3, wherein the first side comprises growth factors, integrins, fibronectin, and/or vitronectin.

5. The composition of claim 4, wherein the growth factors, integrins, fibronectin, and/or vitronectin are 3D printed as the first side.

6. The composition of claim 4, wherein the first side of the mesh is coated with growth factors, integrins, fibronectin, and/or vitronectin.

7. The composition of claim 2, wherein the first side comprises decellularized tissue matrix.

8. The composition of claim 7, wherein the decellularized tissue matrix is 3D printed as the first side.

9. The composition of claim 7, wherein the first side of the mesh is coated with decellularized tissue matrix.

10. The composition of claim 3, wherein the surface of the first side is micropatterned.

11. The composition of claim 10, wherein the micropattern substantially comprises a square, rectangular, triangular, pentagonal, or hexagonal pattern.

12. The composition of claim 3, wherein the second side is substantially smooth and flat.

13. The composition of claim 1, wherein the mesh has a tensile strength of greater than 23 kilopascals (kPa).

14. The composition of claim 1, wherein the mesh is degradable at a rate that is a function of crosslinking of the PVA-P.

15. The composition of claim 1, wherein the thickness of the mesh is in a range of about 100 μm-300 μm, 100 μm-200 μm, 100 μm-150 μm, 100 μm-125 μm, 125 μm-300 μm, 125 μm-200 μm, 125 μm-150 μm, 150 μm-300 μm, 150 μm-200 μm, or 200 μm-300 μm.

16. The composition of claim 1, wherein the mesh has an elastic modulus of greater than 80 kilopascals (kPa).

17. The composition of claim 1, wherein the mesh has a zeta potential of −2.0 millivolts (mV) or less.

18. The composition of claim 1, wherein the mesh comprises antibiotic, anti-inflammatory, and/or analgesic drugs.

19. The composition of claim 18, wherein the mesh comprises controlled release drug delivery nanoparticles further comprising the antibiotic, anti-inflammatory, and/or analgesic drugs.

20. A method of producing the composition of claim 1, comprising the step of manufacturing a mesh comprised of PVA-P or coated with PVA-P.

21. The method of claim 20, wherein the mesh is 3D printed as a network of structures comprising PVA-P.

22. The method of claim 21, wherein a printer deposits a pre-polymer solution that polymerizes to PVA-P during the printing process.

23. The method of claim 21, wherein PVA is mixed with sodium trimetaphosphate solution prior to printing followed by polymer cross-linking during a drying process.

24. The method of claim 20, wherein the first side of the mesh is 3D printed onto the mesh as growth factors, integrins, fibronectin, and/or vitronectin.

25. The method of claim 20, wherein the first side of the mesh is 3D printed onto the mesh as decellularized tissue matrix.

26. The method of claim 20, wherein a mesh comprising a network of structures is coated with PVA-P.

27. A method of repairing a weakness or opening in a tissue of an individual, comprising the step of positioning the composition of claim 1 at the weakness or opening.

28. The method of claim 27, wherein the tissue is a muscular wall.

29. The method of claim 27, wherein the method is used for hernia repair, pelvic floor repair, cardiothoracic surgery repair, tendon repair, or breast implant surgery.

30. The method of claim 27, wherein said composition comprises a first side and a second side and said first side comprises growth factors, integrins, fibronectin, and/or vitronectin.

31. The method of claim 27, wherein said composition comprises a first side and a second side and said first side comprises decellularized tissue matrix.

32. The method of claim 31, wherein the mesh is positioned such that the first side faces the weakness or opening, and the second side faces a direction opposing the wall.

33. The method of claim 27, wherein the opening is a hernia.

34. The method of claim 33, wherein the hernia is diaphragmatic, inguinal, femoral, umbilical, incisional, epigastric, or hiatal.

35. The method of claim 27, wherein there is no adhesion of the composition to one or more peritoneal and/or visceral organs or tissues or wherein there is reduced adhesion to one or more peritoneal and/or visceral organs or tissues compared to when using a composition that lacks PVA-P.

36. The method of claim 27, wherein local inflammation is reduced following placement of the composition.

37. The method of claim 27, wherein the growth factors, integrins, fibronectin, and/or vitronectin stimulates healing of the weakness or opening.

38. The method of claim 27, wherein the decellularized tissue matrix stimulates healing of the weakness or opening.

39. The method of claim 27, wherein the mesh degrades in the individual.

40. The method of claim 39, wherein the mesh degrades in the individual at a controllable rate of degradation.

41. The method of claim 40, wherein the controllable rate of degradation is controlled by the number of phosphate crosslinking groups in the mesh.

42. The method of claim 41, wherein control of sodium trimetaphosphate (STMP) to PVA ratio controls the degree of crosslinking.

43. The method of claim 1, wherein said composition comprises antibiotic, anti-inflammatory, and/or analgesic drugs.

44. The method of claim 43, wherein said composition comprises controlled release drug delivery nanoparticle further comprising the antibiotic, anti-inflammatory, and/or analgesic drugs.
